(19) United States
(12) Patent Application Publication
(10) Pub. No.: US 2026/0295136 A1
(43) Pub. Date: Oct. 1, 2026

(54) DETECTOR FOR DETECTING INCLUSIONS IN FLUID, MEDICAL TREATMENT DEVICE COMPRISING THE SAME, AND METHOD FOR DETECTING INCLUSIONS IN FLUID

(71) Applicant: B. Braun Avitum AG, Melsungen (DE)

(72) Inventors: Rolf Hollstein, Alheim-Heinebach (DE); Johannes Krapf, Bebra (DE); Ernst Bressau, Malsfeld (DE)

(21) Appl. No.: 19/629,494

(22) Filed: Mar. 26, 2026

(30) Foreign Application Priority Data
Apr. 1, 2025 (DE) 10 2025 112 777.8

(51) Int. Cl. A61M 1/36

(52) U.S. Cl. CPC A61M 1/3626; A61M 2205/3375

(57) ABSTRACT

A detector for detecting inclusions in an extracorporeally flowing fluid includes an ultrasound transmitter, ultrasound receiver, and control unit. The control unit drives the ultrasound transmitter with a drive signal. The ultrasound transmitter transmits ultrasound with a transmission energy in accordance with the drive signal through a channel for conducting the fluid. The ultrasound receiver provides a receive signal in accordance with a reception energy of the ultrasound. The control unit adjusts the transmission energy as a function of the receive signal to bring the receive signal to a target value or into a target value interval. The control unit adjusts a number and/or duration of pulses to bring the receive signal to the target value or into the target value interval. The detector can be incorporated into a medical treatment device, and operated in conjunction with a computer-implemented method and/or computer-readable storage medium.

### CROSS-REFERENCE TO RELATED APPLICATIONS

This application claims priority under 35 U.S.C. § 119 to German Application No. 10 2025 112 777.8, filed on Apr. 1, 2025, the content of which is incorporated by reference herein in its entirety.

### FIELD

The present disclosure relates to an ultrasound-based detector for detecting inclusions in flowing fluid, a medical treatment device comprising the same, a computer-implemented method for detecting inclusions in flowing fluid on an ultrasound basis, and a computer-readable storage medium.

### BACKGROUND

In medical treatment devices, in particular dialysis devices, ultrasound-based detectors are typically used to protect patients from dangerous air embolisms. For this purpose, a portion of an extracorporeal blood circuit to be monitored may be conducted through an ultrasound path of the detector in the form of a blood tube. To this end, the detector comprises a transmitting piezoelectric element that, by electrical excitation at its resonant frequency, transmits an ultrasound pulse, or ultrasound waves, through the blood tube. The ultrasound pulse may be received by a receiving piezoelectric element of the detector and converted into a receive signal, or a reception voltage. This is compared by a comparator with a reference voltage and evaluated. On the output side of the comparator, a signal derived from the receive signal is thus available, which provides information as to whether “air” or “no air”, that is to say, only fluid or blood, is present in the ultrasound path.

Tolerances along the ultrasound path lead to tolerances in the coupling and in the sensitivity of the detector with respect to air bubbles. In particular, mention should be made of tolerances in piezo layers and in the potting of the piezoelectric elements, in the housing material or in the geometry of the detector, as well as in the material, in the geometry, in the manufacturing process, or in the sterilization process of the tube used as a single-use product.

An operating region of the detector between the “availability” of a sufficiently strong receive signal that enables reliable detection and the “safety” of not endangering the fluid or the blood to be monitored by an excessively high energy input is comparatively narrow due to the tolerances mentioned.

The aspect of safety is addressed by the publication “Imaging Methods in Medicine. From Technology to Medical Application” by Prof. Dr. Olaf Dössel, Univ. of Karlsruhe, ISBN 3-540-66014-3. It is shown that the ultrasound pulses of the detector damage the patient’s blood conducted through the blood tube when the energy input is excessively high. The damage occurs, in particular, due to thermal effects and due to cavitation. Heat is generated locally in proportion to the sound intensity absorbed by the blood. Cavitation is an effect in which gas bubbles form in tissue during a negative-pressure phase of an ultrasound pulse and then collapse during a pressure phase.

A requirement is thus to ensure the safety of the detector by monitoring and limiting the energy input associated with the ultrasound. For this purpose, patent publication EP 3 070 466 A1 proposes indirectly monitoring the energy input on the basis of the drive signal by which the ultrasound transmitter is driven or excited. In the event of a fault, when the monitoring indicates that the drive signal changes in such a manner that the resulting energy input exceeds a tolerable level, hardware measures of a hydraulic and/or electrical/electronic nature are taken. For example, pumps may be stopped or valves may be closed in order to interrupt the volume flow of the medium or blood. Electrical/electronic measures are aimed at switching off or limiting the energy input. For example, the energy supply to the ultrasound transmitter may be interrupted. Alternatively or additionally, an electrical or electronic component may be dimensioned or selected such that the energy input may be limited thereby, for example, by providing dimensioning or selection of an inductance having a corresponding saturation current, or by providing Zener diodes for voltage limitation.

As already mentioned above, a further requirement is to ensure the availability of the detector, in particular to ensure a sufficient signal strength of the receive signal. Patent publication EP 0 643 301 B1 proposes, for this purpose, comparing a mean value derived from a sequence of the receive signal with a predetermined setpoint and adjusting, as a function of the deviation, an amplitude of the drive signal by which the ultrasound transmitter is driven. In this way, tolerances and aging effects of the ultrasound path are compensated for, and the mean value of the receive signal may be kept constant at a required level.

### SUMMARY

In contrast, the invention is based on the object of providing an ultrasound-based detector for inclusions in flowing fluid by means of which tolerances and aging effects of its ultrasound path may be flexibly compensated for. Furthermore, the invention is based on the object of providing a medical treatment device, a computer-implemented method, and a computer-readable storage medium by means of which tolerances and aging effects of an ultrasound path may be flexibly compensated for.

A basic concept of the disclosure consists of detecting a receive signal of an ultrasound path, evaluating or comparing the receive signal or a signal derived therefrom with a target value or a target value interval, and adjusting, as a function of the evaluation or comparison, a drive signal on which a transmission energy of the ultrasound depends, in order to thus bring the receive signal or the derived signal to the target value or into the target value interval. The drive signal is temporally structured and comprises a temporal sequence of pulses. According to the disclosure, the adjustment is carried out by adjusting the temporal sequence of the pulses, specifically with respect to a duration and/or a number of the pulses. According to the disclosure, tolerances and aging effects in the ultrasound path of the detector, in particular changes in the coupling or attenuation of the ultrasound path, may be flexibly compensated in this way. That is to say, a receive signal or derived signal is provided, the signal strength of which is repeatable and reliable. The quality of the receive signal or derived signal is thus increased.

The quality comprises, in particular, that the receive signal or the derived signal is provided in an optimal operating region of the signal evaluation and/or that an acceptable signal-to-noise ratio is provided, which enables reliable signal analysis.

A detector according to the disclosure is provided for detecting inclusions in an extracorporeally flowing fluid on the basis of ultrasound. In particular, it is provided for detecting bubbles in blood or in a medical solution, in particular for a blood treatment device, in particular a dialysis device, or for an infusion pump. The detector has an ultrasound transmitter, an ultrasound receiver, and a control unit, wherein:
the control unit is configured to drive the ultrasound transmitter with a drive signal that comprises a sequence of pulses,
the ultrasound transmitter is configured to transmit ultrasound having a transmission energy corresponding to the drive signal through a channel which is provided to conduct the fluid,
the ultrasound receiver is configured to receive the ultrasound and to provide a receive signal that corresponds to a reception energy of the ultrasound, and
the control unit is configured, as a function of the receive signal, to adjust the transmission energy in order to bring the receive signal or a signal derived therefrom to a target value or into a target value interval and thus to obtain a receive signal or derived signal, the signal strength of which is repeatable and reliable. The quality of the receive signal or derived signal is thus increased. According to the disclosure, the control unit is configured, within the sequence of the drive signal, to adjust a number or a duration of the pulses, or both, in order to thereby bring the receive signal or the derived signal to the target value or into the target value interval.

Thus, a detector is provided in which tolerances and aging effects of its ultrasound path may be very flexibly compensated for:
by adjusting the duration of one or more pulses of the sequence,
by adjusting the number of pulses of the sequence, or
by adjusting both the duration and the number.

Additionally, the adjustment may be carried out by adjusting an amplitude of the ultrasound transmission pulses.

According to a further development, the control unit is configured to compare the receive signal with a reference value and, as a function of the comparison, to output an output signal that indicates a detection or identification result with respect to inclusions, preferably quantified.

According to a further development, the ultrasound transmitter and the ultrasound receiver are each formed by a piezoelectric element, and an inductance is provided as a voltage device electrically in parallel with the ultrasound transmitter.

The sequence of pulses of the drive signal according to the disclosure preferably comprises a charge pulse having a charging duration or charging time, during which the inductance is connected to a voltage source and is charged.

In the sequence of pulses, according to the disclosure, the charge pulse is followed by a number of excitation pulses for exciting the ultrasound transmitter.

Preferably, the sequence of pulses and a subsequent pause define a burst interval of the drive signal. The burst interval thus defines the time span between start times of successive charge pulses, or of the drive signal.

Preferably, the control unit is configured to drive the ultrasound transmitter with a sequence of drive signals, or burst intervals. This results in a corresponding sequence of transmitted ultrasound signals and a corresponding sequence of receive signals.

Preferably, the signal derived from the receive signal is a statistical value or a statistical quantity, in particular, a median, a mean value, a maximum value, and/or a standard deviation of the sequence of receive signals.

According to a further development, the control unit comprises a correlation of the number and/or the duration, and, in particular, of the resulting transmission energy, as a function of at least one parameter of the detector, of the channel, and/or of the fluid. In particular, the correlation comprises a look-up table and/or an algorithm. By means of the correlation, the control unit according to the disclosure comprises suitable initial values or start values for the number and/or the duration, by which the receive signal or the signal derived therefrom may initially already be brought as close as possible to the target value or into the target value interval, such that subsequent adjustments of the number and/or the duration are minimized or may even be omitted.

Preferably, the control unit is configured, with knowledge of the at least one parameter, for example, after its input, detection, or ascertainment, to initially select the number or the duration, or both.

The attribute initial, within the scope of the disclosure, stands, in particular, for a time of commissioning of the detector or for a change of the channel, the fluid, or an operating mode of the detector.

A change exists, for example, when the operating mode changes from priming to therapy, or vice versa, or when the fluid to be monitored changes, for example, from priming fluid to blood, or vice versa, or when the tube is changed.

With each change, the effect may occur that the coupling and/or the attenuation of the ultrasound changes. The above-mentioned correlation maps this change empirically, such that the number and/or the duration initially selected after the change initially already compensates for the change or at least partially compensates for it.

According to a further development, the above-mentioned at least one parameter is a manufacturer designation, type designation, ID, electrical or geometric nominal variable, or a material of the ultrasound transmitter, of the ultrasound receiver, or of the channel, or it is a volume flow or a type of the fluid, for example, “priming fluid” or “medical solution” or “blood”, or an operating mode of the detector, for example, “in treatment” or “in preparation of a treatment”.

In the case of an adjustment in the sense of increasing the charging duration of the charge pulse, the current of the inductance approaches the saturation current, as a result of which an increasingly stronger magnetic field results and, consequently, the self-induced voltage of the inductance is higher. When the inductance is discharged via the subsequent excitation pulses, the transmission energy is increased in accordance with the higher self-induced voltage of the inductance.

In the same direction, namely in the direction of increasing the transmission energy, an increase in the number of excitation pulses is effective.

The respective opposite reduction in the sense of a reduction of the charging duration and/or of the number of excitation pulses leads to a reduction of the transmission energy.

The charging duration is preferably about 15μs ± 5μs. The frequency of the excitation pulses—for exciting the ultrasound transmitter at its resonant frequency—is preferably about 1 to 5 MHz, more preferably about 2 MHz. A pause duration following the sequence of pulses and until the next charge pulse is preferably about 300μ s to 500 μ s, preferably about 480μ s. The time span of the drive signal, or burst interval, that is to say the time span of the sequence of pulses including the subsequent pause, is preferably about 50 to 900 μs, preferably about 500 μs. A frequency of the burst interval is preferably 1 to 15 kHz, more preferably about 2 kHz.

Preferably, the ultrasound transmitter and the ultrasound receiver are arranged on an axis that extends through a center of the container. Thus, the ultrasound path is short, the detector is compact, and the attenuation is low, such that a comparatively strong receive signal is already possible with comparatively low transmission energy.

Preferably, the ultrasound transmitter and the ultrasound receiver form between them a receptacle into which the channel or the tube may be inserted and positionally fixed.

Preferably, a peak value rectifier is provided in which the receive signal is processed.

Preferably, a comparator is provided into which the output signal of the peak value rectifier and a reference value are input, wherein a comparator output signal is a pulse width of a rectangular signal that is formed from the receive signal and the reference value.

Preferably, a mean value forming unit is provided, by means of which previously ascertained pulse widths are combined to form a mean value.

An output signal of the mean value forming unit is the above-mentioned mean value of the sequence of receive signals.

According to a further development, the control unit is configured to periodically form the receive signal, preferably the derived signal, more preferably the mean value, to compare it with the target value or the target value interval, and to adjust the number or the duration, or both, in particular, when the receive signal, preferably the derived signal, more preferably the mean value, leaves the target value interval. Thus, the transmission energy is adjusted only when the mean value of the receive signal leaves the target value interval. The receive signal, the derived signal, or the mean value may thus drift within the target value interval without the transmission energy being adjusted, which reduces the effort.

According to a further development, the control unit is configured to adjust the number or the duration, or both, in particular, with a predetermined step size, until the receive signal, the derived signal, or the mean value is brought to the target value or into the target value interval.

According to a further development, the control unit is configured to increase the number or duration or both when the receive signal, the derived signal, or the mean value is below the target value or a lower limit of the target value interval, and to reduce the number or duration or both when the receive signal, the derived signal, or the mean value is above the target value or an upper limit of the target value interval.

According to a further development, the control unit is configured to compare the number or the duration, or both, with a respective predetermined upper limit and to increase them only when they are below their respective predetermined upper limit or limits. Thus, it is ensured that an upper limit of the transmission energy is not exceeded and the fluid is not damaged.

Alternatively or additionally, the control unit is configured to compare the number or the duration or both with a respectively predetermined lower limit and to reduce the number or the duration or both only when the number or the duration or both are above their respectively predetermined lower limit. Thus, it is ensured that a lower limit of the transmission energy is not undershot in order to ensure that a sufficiently available receive signal is provided.

According to a possible further development, the control unit is configured to form a deviation of the receive signal, the derived signal, or the mean value from the target value, wherein the control unit comprises a control device and a controlled variable of the control device is the mean value, a setpoint variable of the control device is the target value, and manipulated variables of the control device are selectively the number or the duration, or both.

According to the disclosure, a medical treatment device, in particular, a dialysis device, has a detector that is configured according to at least one aspect of the preceding description, wherein a channel through which the fluid is flowable, or through which the fluid flows, in particular a tube, is arranged between the ultrasound transmitter and the ultrasound receiver.

A method according to the disclosure for detecting inclusions, in particular, bubbles, in an extracorporeally flowing fluid, in particular in blood or a medical solution, is provided for an ultrasound-based detector. In particular, the detector is configured according to at least one aspect of the preceding description and is provided on a medical treatment device, in particular on a dialysis device. The method according to the disclosure has the following steps:
driving an ultrasound transmitter of the detector with a drive signal that comprises a sequence of pulses by means of a control unit,
transmitting ultrasound having a transmission energy corresponding to the drive signal through a channel conducting the fluid, in particular, a tube by means of the ultrasound transmitter,
receiving the ultrasound and providing a receive signal in accordance with a reception energy of the ultrasound by means of an ultrasound receiver of the detector,
preferably forming a signal derived from the receive signal, more preferably forming a mean value of the receive signal as the derived signal, and
adjusting the transmission energy as a function of the receive signal, in order to bring the receive signal, preferably the signal derived therefrom, more preferably the mean value, to a target value or into a target value interval by means of the control unit. According to the disclosure, the adjustment of the transmission energy is carried out by adjusting a number and/or a duration of the pulses of the drive signal.

According to a further development, the method has steps:
reading at least one parameter of the detector, the channel, or the fluid by means of the control unit,
accessing a correlation, in which an initial number and/or an initial duration, in particular an initial transmission energy, is or are stored as a function of the at least one parameter by means of the control unit,
selecting the initial number and/or the initial duration, and
driving the ultrasound transmitter with the drive signal having the initial number and/or the initial duration, in order to initially bring the receive signal, preferably the signal derived therefrom, more preferably the mean value, as close as possible to the target value or the target value interval by means of the control unit.

According to a further development of the method, the receive signal, the derived signal, or the mean value is periodically compared with the target value or the target value interval, and the number or the duration, or both, are adjusted.

According to a further development of the method, the adjustment of the number or the duration, or both, is carried out, in particular, with a predetermined step size, until the receive signal, the derived signal, or the mean value is brought to the target value or into the target value interval.

According to a further development of the method, the receive signal, the derived signal, or the mean value is periodically compared with the target value interval, and the adjustment of the number or the duration, or both, is carried out as soon as the comparison indicates that the receive signal, the derived signal, or the mean value leaves the target value interval.

According to a further development of the method, the number or the duration, or both, are increased when the receive signal, the derived signal, or the mean value lies below the target value or a lower limit of the target value interval, and the number or the duration, or both, are reduced when the receive signal, the derived signal, or the mean value lies above the target value or an upper limit of the target value interval.

According to a further development, the method has steps:
comparing the number or the duration or both with a respectively predetermined upper limit, and
increasing the number or the duration or both only when the number or the duration or both are below their respectively predetermined upper limit by means of the control unit, and
comparing the number or the duration or both with a respectively predetermined lower limit, and
reducing the number or the duration or both only when the number or the duration or both are above their respectively predetermined lower limit by means of the control unit.

### BRIEF DESCRIPTION OF THE DRAWINGS

In the following, preferred embodiments of the invention are explained in greater detail with reference to the drawings, of which:

FIG. 1, in a schematic representation, shows a medical treatment device according to the disclosure having a detector according to the disclosure having an ultrasound transmitter and an ultrasound receiver, as well as having a control unit;

FIG. 2, in a schematic representation, shows a drive signal for the ultrasound transmitter according to FIG. 1;

FIG. 3, in a schematic representation, shows the drive signal according to FIG. 2 with a charge pulse having an adjusted charging duration;

FIG. 4, in a schematic representation, shows the drive signal according to FIG. 2 having an adjusted number of excitation pulses;

FIG. 5, in a schematic representation, shows the drive signal according to FIG. 2 and a receive signal of the ultrasound receiver resulting therefrom;

FIG. 6, in a schematic representation, shows the drive signal according to FIG. 5 having a higher number of excitation pulses and the receive signal of the ultrasound receiver resulting therefrom;

FIG. 7, in a schematic representation, shows a block diagram of the detector according to FIG. 1 with a feedback and evaluation of a signal derived from the receive signal and an adjustment of the charging duration of a charge pulse and/or of the number of excitation pulses of the drive signal;

FIG. 8 shows, in a schematic representation, shows three different profiles of the receive signal that result, for a given drive signal and a given fluid flow, from different degrees of attenuation/coupling of the ultrasound path;

FIG. 9 shows, in a schematic representation, shows a flow diagram of a method according to the disclosure with an adjustment of the number of excitation pulses of the drive signal for the detector; and

FIG. 10 shows, in a schematic representation, shows a flow diagram of a method according to the disclosure with an adjustment of the charging duration of the charge pulse of the drive signal for the detector.

### DETAILED DESCRIPTION

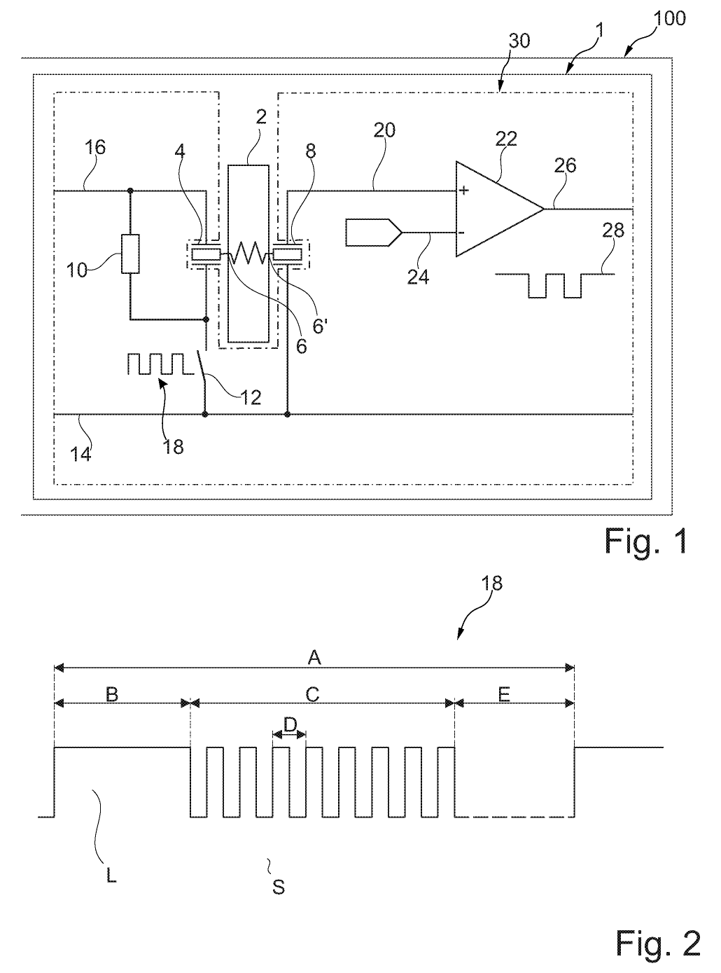

According to FIG. 1, a detector 1 is provided for detecting air in a fluid, in the form of blood of a dialysis patient. The detector 1 is a component of a medical treatment device 100 configured as a dialysis machine, which is illustrated only in a very rough schematic manner. Blood flows through a tube 2, and the detector 1 comprises, each in the form of a piezoelectric element, an ultrasound transmitter 4 for transmitting ultrasound 6 through the tube 2, and an ultrasound receiver 8 for receiving ultrasound. The ultrasound transmitter 4, the tube 2 containing the fluid, and the ultrasound receiver 8 form an ultrasound path. To transmit the ultrasound, the ultrasound transmitter 4 is supplied with an electrical voltage via an inductance 10 arranged electrically in parallel with the ultrasound transmitter 4.

The ultrasound transmitter 4 and the inductance 10 are electrically connectable in parallel to a ground 14 via a switch 12. Furthermore, they are connected electrically in parallel to a voltage source 16. A self-induced voltage of the inductance 10 is generated when the switch 12 is opened and is then applied to the ultrasound transmitter 4. High-frequency opening and closing of the switch 12 thus leads to a high-frequency voltage change at the ultrasound transmitter 4 and to a corresponding ultrasound 6. This driving and thus excitation of the ultrasound transmitter 4 is carried out in accordance with a drive signal 18 of a control unit 30 of the detector 1.

The transmitted ultrasound 6, which has passed through the tube 2 and the fluid flowing therein, is received by the ultrasound receiver 8 and converted into an electrical receive signal 20. The receive signal 20 is compared with and evaluated against a reference value/a reference voltage 24 by a comparison unit 22. At an output 26 of the comparison unit 22, a output signal 28 derived from the receive signal 20 is thus available, which provides at least qualitative information as to whether “air” or “no air” is present in the ultrasound path. Quantitative statements regarding the air volume may also be derived.

In FIG. 2, the drive signal 18 is shown over time. The drive signal 18 has a temporal structure, that is to say the voltage of the drive signal 18 varies as a function of time, such that it has a sequence of pulses L, S. The drive signal 18 is structured into burst intervals A. One of the burst intervals A lasts, for example, between 50 and 900 μs. A repetition frequency of the burst intervals A is, for example, 1 to 15 kHz.

The burst interval A itself comprises, according to FIG. 2, three successive sections with different voltage profiles: First, a charge pulse L with a charging duration B, during which the switch 12 is closed, such that the voltage of the voltage source 16 from FIG. 1 is applied to the inductance 10 and the inductance 10 is charged. The charging duration B of the inductance 10 is, for example, 5 to 20 μs. After expiration of the charging duration B, a number C of excitation pulses S follows with an excitation frequency D of the ultrasound transmitter 4 of, for example, 1 to 5 MHz. Here, according to FIG. 2, for example, eight excitation pulses S are provided. A spacing between two excitation pulses S is, for example, in the case of an excitation frequency of 2 MHz, 500 ns. After expiration of the excitation pulses S, a pause E without excitation pulses and without charging of the inductance 10 follows until the charging duration B of a new burst interval A starts. The pause E is, for example, 300 to 500 μs, for example, 481 μs. The burst interval A is thus the spacing between two start times of successive charge pulses L, or charging durations B.

With respect to a burst interval A, a magnitude or an energy of the ultrasound 6 generated therewith increases both with the charging duration B of the charge pulse L for the inductance 10 and with the number C of excitation pulses S. Conversely, the strength or energy of the ultrasound 6 decreases with decreasing charging duration B and/or number C.

A change in the charging duration B or the number C, or both, may thus be used according to the disclosure to change or adjust the transmission energy of the ultrasound 6 transmitted by the ultrasound transmitter 4 in order to compensate for tolerances in the coupling and/or attenuation of the ultrasound path.

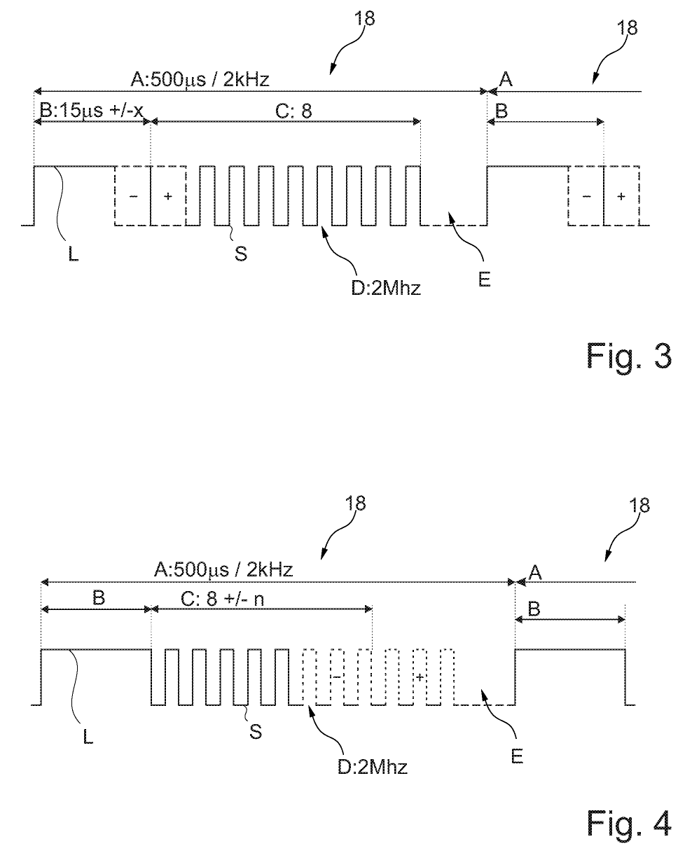

FIG. 3 shows, by way of example, a possible change of the drive signal 18, and thus of the transmission energy of the ultrasound 6, by changing the charging duration B from 15 μs by ± x μs, with the pause E, the frequency D, and the number C of the excitation pulses S otherwise remaining unchanged.

FIG. 4 shows the possibility of increasing and decreasing the number C by, for example, three excitation pulses S in each case, starting from eight excitation pulses S, and thus a corresponding change or adjustment of the transmission energy of the ultrasound 6 resulting from the drive signal 18, with the frequency D, the charging duration B, and the duration of the burst interval A, or of the drive signal 18, otherwise remaining unchanged.

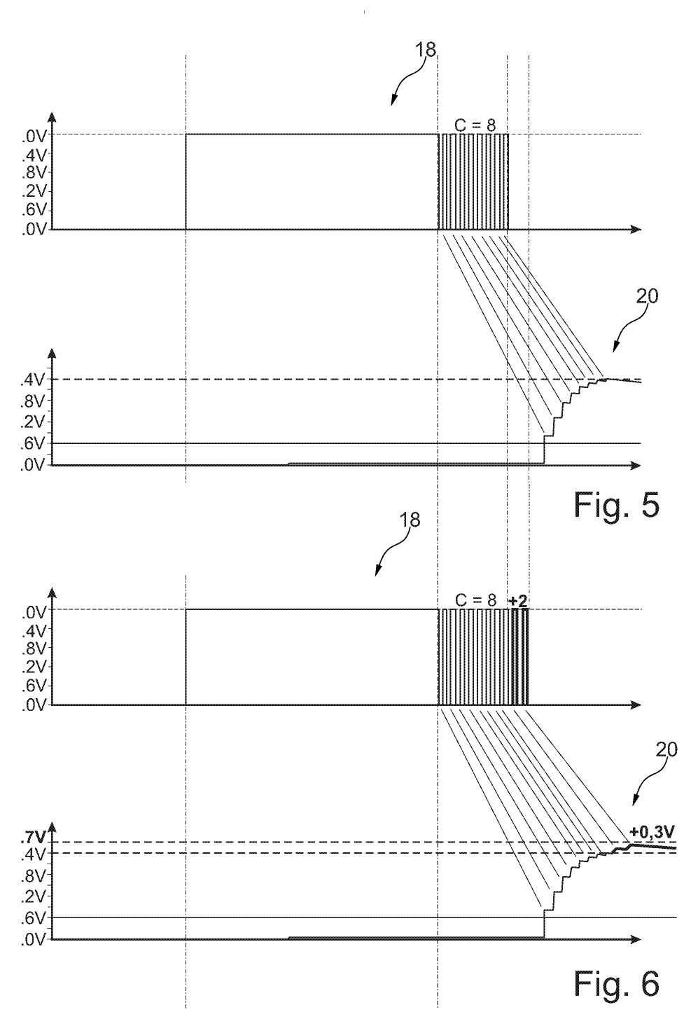

FIG. 5 at the top shows the drive signal 18 with eight excitation pulses S as a function of time. FIG. 5 at the bottom shows the receive signal 20 provided by the ultrasound receiver 8, based on the ultrasound received 6′, as a function of time. It is illustrated that each excitation pulse S of the burst signal A illustrated above further increases the transmission energy of the ultrasound 6 and thus also a reception energy of the receive signal 20 illustrated at the bottom of FIG. 5. The strength of the receive signal 20 achieved according to FIG. 5 with the charging duration B and the number C of eight excitation pulses S is 2.4 V.

FIG. 6 shows an adjusted drive signal 18 based on FIG. 5. The adjustment consists in that the number C of the excitation pulses S has been increased from eight to ten. As already explained, this leads to stronger ultrasound 6. According to FIG. 6 below, the achieved strength of the receive signal 20 is thereby also increased from 2.4 V to 2.7 V, that is to say, by +0.3 V.

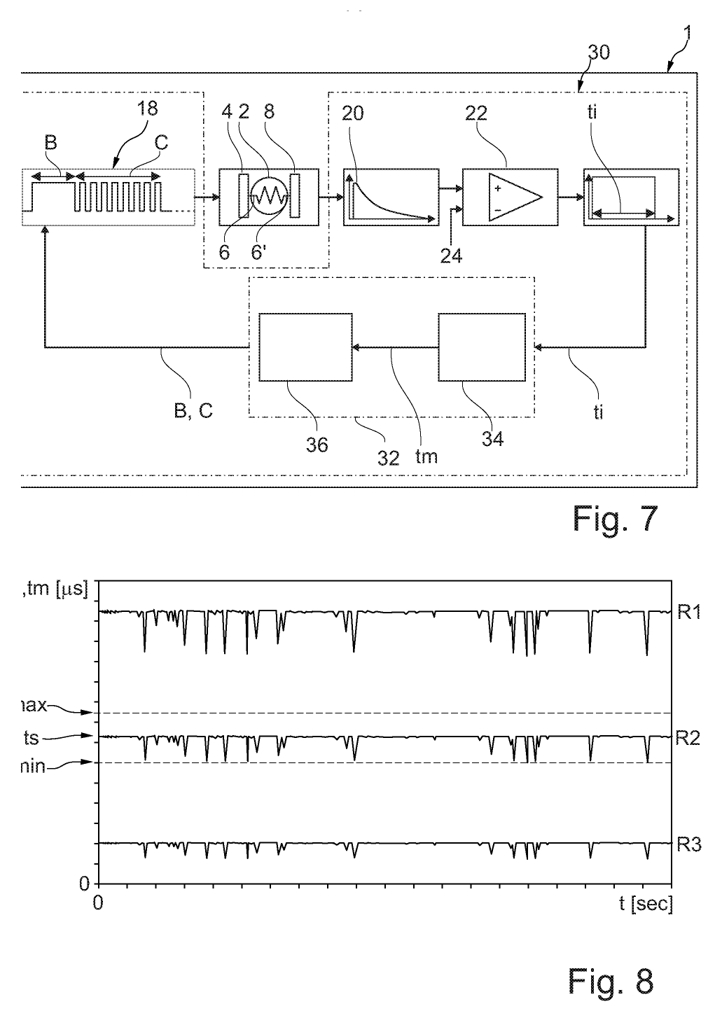

FIG. 7 shows, with reference to FIGS. 1 and 2, in a schematic representation, a block diagram of the detector 1. The starting point is the drive signal 18 according to FIG. 2 having the previously described sequence of pulses and the charging duration B of the charge pulse L and the number C of the excitation pulses S. During the charging duration B, charging of the inductance 10 according to FIG. 1 takes place. Following the charging duration B, the high-frequency discharge of the inductance 10 according to FIG. 1 takes place with the number C of excitation pulses S. Accordingly, the ultrasound transmitter 4 is excited at its resonance frequency and transmits ultrasound 6 with a transmission energy corresponding to the drive signal 18. The ultrasound passes through the tube 2 with flowing fluid and, depending on the quality of the coupling, on material properties, on the flow, and, if applicable, on other parameters, undergoes attenuation. The attenuated ultrasound 6′ impinges on the ultrasound receiver 8 with a reception energy that is lower than the transmission energy. The latter provides the receive signal 20 corresponding to the reception energy, which is supplied to a peak value rectifier. Subsequently, the signal is applied to the comparator 22, which carries out the comparison with the reference value or the reference voltage 24. A signal derived from the receive signal 20 results in the form of a square-wave signal, the positive pulse width ti of which is detected.

In the illustrated exemplary embodiment, the above-described driving of the ultrasound transmitter 4 with the drive signal 18 is carried out periodically at the repetition frequency of 2 kHz of the drive signal 18, that is to say, every 500 s of the burst interval A, wherein, each time, the above-mentioned positive pulse width ti of the receive signal 20 is detected.

A sequence of the repeatedly detected positive pulse widths ti is supplied to a microcontroller 32 of the control unit 30.

An evaluation unit 34 of the microcontroller 32 forms a statistical variable from the positive pulse widths ti of the sequence.

In the exemplary embodiment shown, this statistical variable is an average or mean value tm. In other words, the evaluation unit 34 derives or forms the mean value tm as a statistical variable from a sequence of receive signals 20.

An adjustment unit 36 of the microcontroller 32 checks whether this mean value tm lies within a predetermined or desired target value interval tsmin, tsmax. If this is not the case, the charging duration B of the charge pulse L or the number C of the excitation pulses S of the drive signal 18 is adjusted, or the charging duration B and the number C are adjusted. From this adjustment, a corresponding change or adjustment of the transmission energy introduced into the ultrasound path 4, 2, 8 results, which in turn leads to a changed or adjusted receive signal 20 and, consequently, to a changed or adjusted mean value tm. The adjustment according to the disclosure is terminated when the check by the adjustment unit 36 indicates that the mean value tm has been brought into the target value interval tsmin, tsmax.

To illustrate the influence of tolerances of the ultrasound path 4, 2, 8, three signal profiles are illustrated by way of example in FIG. 8, which result, for a given drive signal 18 and fluid flow, for different tolerances and thus differently strong attenuations of the ultrasound path 4, 2, 8. Illustrated is the temporal signal profile of the positive pulse width ti. It can be seen that, during a passage of bubbles, the positive pulse width ti decreases in each case. The larger the bubble, the stronger the reduction.

For example, a low attenuation of the ultrasound 6 along the ultrasound path results in a strong signal profile of the positive pulse width ti, for example, in the region R1. In region R2, in contrast, a medium attenuation is present, and in region R3 the strongest attenuation is present, which, compared to region R1, leads to correspondingly attenuated signal profiles of the positive pulse width ti and also to correspondingly attenuated drops.

According to FIG. 8, region R2 is the region in which the mean value tm of the receive signal 20 lies within the target value interval tsmin, tsmax. If the mean value tm exceeds or meets the upper limit tsmax, the adjustment described above according to the disclosure is carried out in the sense of a reduction of the charging duration B and/or a reduction of the number C. In the reverse case, if the mean value tm meets or falls below the lower limit tsmin, the adjustment described above is carried out in the sense of an increase of the charging duration B and/or an increase of the number C.

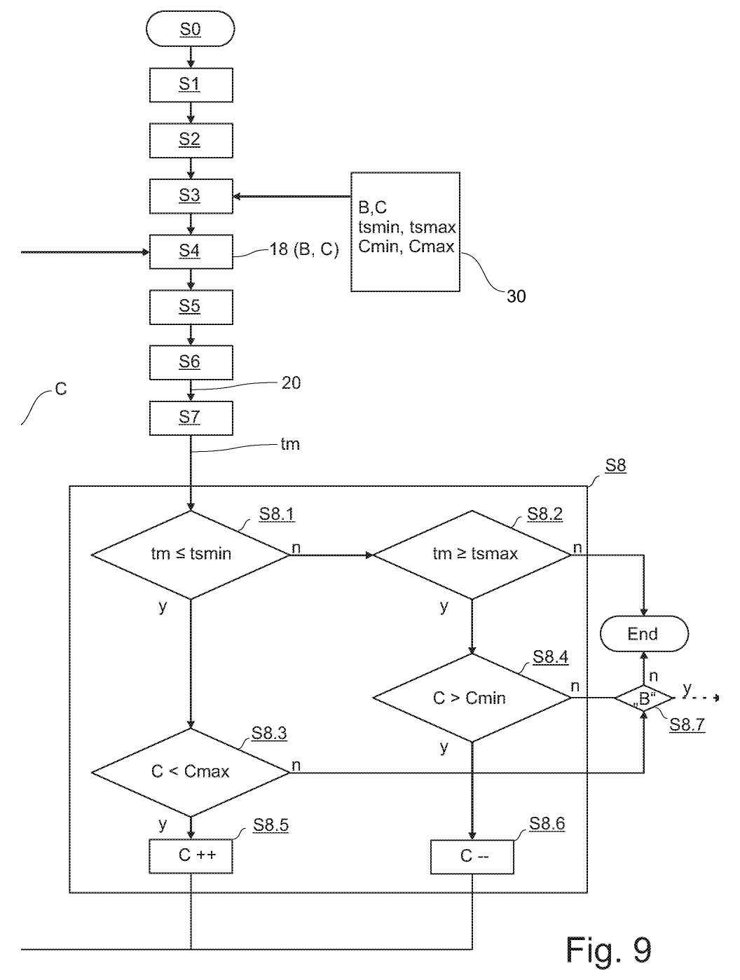

FIG. 9 shows, in a schematic representation, a flow diagram of a method for detecting inclusions in an extracorporeally flowing fluid for the ultrasound-based detector 1, in which this adjustment of the transmission energy/the drive signal 18 is carried out according to the disclosure. The description of FIG. 9 is also provided with reference to FIG. 7.

Initially, the step Start or initialization S0 of the method is carried out, for example, by a corresponding manual input into a user interface and/or by detection of the tube 2 inserted between the ultrasound transmitter 4 and the ultrasound receiver 8.

This is followed by the step S1 of reading at least one parameter that characterizes the detector 1, the channel 2, the fluid, and/or a fluid flow, for example, by manual input into a user interface, by detection via a detection unit, or by ascertainment.

This is followed by the step of accessing S2 a correlation 30, in particular, an empirically ascertained correlation. In the correlation, depending on the at least one parameter, an initial number C of excitation pulses S and/or an initial duration B of the charge pulse L is stored.

In the exemplary embodiment shown, the correlation 30 comprises parameters of the ultrasound path, which have an influence on the attenuation of the ultrasound and characterize the flowing fluid. Examples thereof include: a type of operating mode of the detector 1 or of the treatment machine 100, for example, priming, blood treatment, or infusion; a type of the fluid to be monitored, for example, priming fluid, blood, or a medical solution; a volumetric flow rate of the fluid; a type, a nominal variable, and a material of the tube; and the like.

The correlation 30 moreover comprises, in the illustrated exemplary embodiment, in dependence on the at least one parameter, the lower limit and the upper limit of the transmission energy, allocated to the charging duration B of the charge pulse L and the number C of the excitation pulses S. These limits are Bmin and Bmax for the limits of the charging duration B and Cmin and Cmax for the limits of the number C of the excitation pulses S.

Moreover, in the exemplary embodiment shown, the correlation 30 comprises the lower and upper limit tsmin, tsmax of the target value interval of the derived signal or of the mean value tm, as a function of the at least one parameter, in particular, as a function of the operating mode, the fluid, and the fluid flow.

This is followed by the step of selecting S3 the initial number C of the excitation pulses S and/or the initial duration B of the charge pulse L as a function of the at least one parameter.

This is followed by the step of driving S4 the ultrasound transmitter 4 using the drive signal 18 having the initial number C and/or the initial duration B, which leads to excitation of the ultrasound transmitter 4 at its resonant frequency.

This is followed by the step of transmitting S5 the ultrasound 6 having the transmission energy corresponding to the drive signal 18 from the ultrasound transmitter 4, through the tube 2 and the fluid, to the ultrasound receiver 8.

The ultrasound receiver 8 receives and converts the ultrasound 6′ into the receive signal 20, and this is followed by the step of providing S6 the receive signal 20 in accordance with the reception energy of the ultrasound received 6′.

This is followed by processing of the receive signal 20 by the peak value rectifier and comparison of this signal with the reference value 24 by the comparator 22. As already mentioned, the comparator 22 outputs the positive pulse width ti for the respective receive signal 20.

After expiration of a sequence of drive signals 18, or burst intervals A, respectively, the step of forming S7 the signal tm derived from the receive signal 20 by the evaluation unit 34 according to FIG. 7 follows. Generally expressed, this is achieved by a statistical evaluation of the positive pulse widths ti of the sequence of receive signals 20.

The statistical evaluation may, in particular, comprise forming a statistical quantity, in particular, a mean value, a median, a maximum value, and/or a standard deviation.

In the specific exemplary embodiment according to FIG. 9, the mean value tm is formed as a statistical variable or as a derived signal.

Subsequently, it must be checked whether the mean value tm derived as described above already lies within the target value interval tsmin, tsmax and, accordingly, no further adjustment of the transmission energy is required, or whether it lies outside the target value interval tsmin, tsmax and, accordingly, the number C of the excitation pulses S and/or the charging duration B of the charge pulse L must be adjusted.

This check is carried out within the step of adjusting S8 the transmission energy in order to bring the mean value tm into the target value interval tsmin, tsmax.

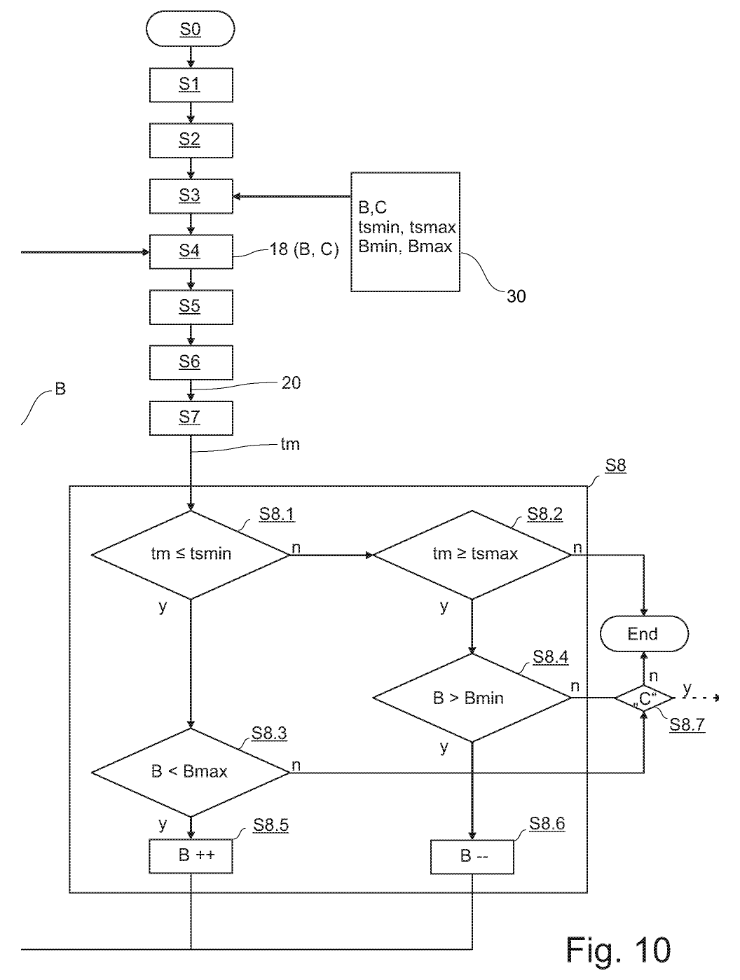

Step S8 comprises, for this purpose, a sequence of steps S8.1 to S8.6, which, for the sake of clarity, is carried out and described below solely by means of the number C of the excitation pulses S. The sequence of steps S8.1 to S8.6 may alternatively or additionally be carried out with the charging duration B of the charge pulse L and its above-mentioned limits Bmin, Bmax, as illustrated in FIG. 10.

In the exemplary embodiment shown in FIGS. 9 and 10, the target value interval tsmin, tsmax is defined such that its lower limit tsmin and its upper limit tsmax are not included.

First, the step of querying S8.1 is carried out as to whether the mean value tm is less than or equal to the lower limit tsmin loaded from the correlation 30. If the lower limit tsmin were to be included, it would be queried here whether the mean value tm is less than the lower limit tsmin.

If the query S8.1 yields that the mean value tm is greater than the lower limit tsmin, the step of querying S8.2 is carried out as to whether the mean value tm is greater than or equal to the upper limit tsmax loaded from the correlation 30. If the upper limit tsmax were to be included, it would be queried here whether the mean value tm is greater than the upper limit tsmax.

If this query S8.2 yields that the mean value tm is less than the upper limit tsmax, the mean value tm consequently already lies within the target value interval tsmin, tsmax. The transmission energy thus already lies within its limits with the previously initially selected values of the number C of the excitation pulses and of the charging duration B, and the step of adjusting S8 the transmission energy may be terminated with the step End.

If, in contrast, the above-mentioned query S8.1 yields that the mean value tm is less than or equal to the lower limit tsmin, this can be changed, or the mean value tm can be brought into the target value interval tsmin, tsmax, by increasing the number C of the excitation pulses S.

However, it must first be checked whether this increase of the number C of the excitation pulses S is permissible. For this purpose, query S8.3 is carried out as to whether the number C of the excitation pulses S is less than its upper limit Cmax loaded from the correlation 30.

If this query S8.3 yields that the number C of the excitation pulses S is greater than or equal to the upper limit Cmax, this means that the number C of the excitation pulses S cannot be increased any further.

The adjustment of the transmission energy on the basis of the number C of the excitation pulses S is terminated.

However, since the mean value tm still lies below its lower limit tsmin of the above step S8.1), the further necessary increase of the transmission energy of the drive signal 18 may subsequently optionally or additionally be carried out by increasing the charging duration B of the charge pulse L. Therefore, a query S8.7 is carried out as to whether the adjustment of the transmission energy on the basis of adjusting the charging duration B of the charge pulse L is to be continued.

The adjustment of the charging duration B may be carried out in comparatively small steps, quasi-continuously. In contrast, the adjustment of the number C of the excitation pulses S may be carried out in integer steps, and thus comparatively large steps. This difference in the step sizes may be advantageously utilized. For example, the adjustment of the charging duration B may be used in a period up to a next adjustment of the number C of the excitation pulses S in order to increase or reduce the transmission energy of the drive signal 18 in small steps, quasi-continuously. Additionally or alternatively, the drive signal 18 may be adjusted, depending on the distance of the derived signal or of the mean value tm from the target value interval tsmin, tsmax, with a large step size by adjusting the number C of the excitation pulses S, or with a fine, quasi-continuous step size by adjusting the charging duration B. Additionally or alternatively, the adjustment of the charging duration B may be used to adjust the derived signal or the mean value tm within the target value interval tsmin, tsmax or close to its limits tsmin, tsmax, in particular when the target value interval is very narrow.

If, however, the above-mentioned query S8.3 yields that the number C of the excitation pulses S is less than its upper limit Cmax, the number C of the excitation pulses S may be further increased in order thereby to increase the transmission energy and thus the mean value tm.

Accordingly, the number C of the excitation pulses S is increased by a defined step in step S8.5, and the new or increased number C of the excitation pulses S is again supplied to the step of driving S4 the ultrasound transmitter 4 with the drive signal 18 having the number C.

If the above-mentioned query S8.2 yields that the mean value tm is greater than or equal to the upper limit tsmax, the mean value tm consequently does not yet lie within the target value interval tsmin, tsmax, but rather above it.

The transmission energy underlying the mean value tm could thus be reduced by reducing the number C of the excitation pulses S in order to bring the mean value tm into the target value interval tsmin, tsmax. For this purpose, it must first be checked whether the number C of the excitation pulses S lies above its lower limit Cmin drawn from the correlation 30, which is carried out in the query step S8.4.

If this query S8.4 yields that the number C of the excitation pulses S is less than or equal to its lower limit Cmin, this means that the number C of the excitation pulses S cannot be reduced any further.

The adjustment of the transmission energy on the basis of the number C of the excitation pulses S is terminated, and a query S8.7 is carried out as to whether the adjustment of the transmission energy on the basis of adjusting the charging duration B of the charge pulse L is to be continued.

However, since the mean value tm still lies above its upper limit tsmax of step S8.2), the further necessary reduction of the transmission energy may subsequently optionally be carried out by reducing the charging duration B of the charge pulse L.

If, however, this query S8.4 yields that the number C of the excitation pulses S is greater than its lower limit Cmin, the number C of the excitation pulses S may be further reduced in order thereby to reduce the transmission energy of the drive signal 18 and thus the mean value tm.

Accordingly, the number C of the excitation pulses S is reduced by a defined step in step S8.6, and the new or reduced number C of the excitation pulses S is again supplied to the step of driving S4 of the ultrasound transmitter 4 with the drive signal 18 having the number C.

The adjustment of the number C of the excitation pulses S and/or of the charging duration B by means of steps S8.1 to S8.6 is carried out until the mean value tm has been brought into the target value interval tsmin, tsmax.

FIG. 10 shows, alternatively or additionally to the method according to FIG. 9, the adjustment of the transmission energy of the drive signal 18 by adjusting the charging duration B of the charge pulse L of the drive signal 18. The two adjustments by means of the number C of the excitation pulses S and by means of the charging duration B are linked via query S8.7.

Disclosed is a detector for detecting inclusions in a flowing fluid, in particular in a tube by means of ultrasound, in which tolerances and attenuations of the ultrasound signal may be compensated by a variable adjustment of a transmission power or transmission energy of the ultrasound signal by adjusting or controlling the charging duration of an inductance and/or by adjusting or controlling the number of excitation pulses.

### LIST OF REFERENCE SIGNS

1 detector
2 tube
4 ultrasound transmitter
6 ultrasound transmitted
6' ultrasound received
8 ultrasound receiver
10 inductance
12 switch
14 ground
16 voltage source
18 drive signal
20 receive signal
22 comparator
24 reference value
26 output
28 output signal
30 control unit
32 microcontroller
34 evaluation unit
36 adjustment unit
100 medical treatment device
A burst interval
L charge pulse
B charging duration
S excitation pulse
C number of excitation pulses
D ultrasound excitation frequency
E pause
ti positive pulse width
tm derived signal or mean value
ts target value
tsmin lower limit of the target value interval
tsmax upper limit of the target value interval
S0 start
S1 reading parameter(s)
S2 accessing correlation
S3 selecting number or duration
S4 driving ultrasound transmitter
S5 transmitting ultrasound
S6 providing receive signal
S7 forming derived signal or mean value
S8 adjusting number and/or duration
S8.1 comparing lower limit of target value interval
S8.2 comparing upper limit of target value interval
S8.3 comparing upper limit of number or duration
S8.4 comparing lower limit of number or duration
S8.5 increasing number or duration
S8.6 reducing number or duration
S8.7 query change of the adjustment

1. A detector for detecting inclusions in a fluid flowing extracorporeally, the detector comprising:
an ultrasound transmitter;
an ultrasound receiver; and
a control unit,
the control unit being configured to drive the ultrasound transmitter with a drive signal that comprises pulses in a sequence,
the ultrasound transmitter being configured to transmit ultrasound with a transmission energy in accordance with the drive signal through a channel conducting the fluid,
the ultrasound receiver being configured to provide a receive signal in accordance with a reception energy of the ultrasound, and
the control unit being configured to adjust the transmission energy as a function of the receive signal in order to bring the receive signal to a target value or into a target value interval,
the control unit being further configured to adjust a number of and/or a duration of pulses of the drive signal in order to bring the receive signal to the target value or into the target value interval.

2. The detector according to claim 1, wherein a signal derived from the receive signal is a statistical variable of a sequence of successive receive signals.

3. The detector according to claim 2, wherein the statistical variable is a mean value, a median, a maximum value, and/or a standard deviation.

4. The detector according to claim 1, wherein the control unit comprises a correlation of the number of and/or the duration of pulses, as a function of at least one parameter of the detector and/or of the channel and/or of the fluid, and the control unit is configured to initially select the number of and/or the duration of pulses as a function of the at least one parameter in order to initially bring the receive signal as close as possible to the target value or the target value interval.

5. The detector according to claim 4, wherein the correlation comprises a look-up table and/or an algorithm.

6. The detector according to claim 4, wherein the at least one parameter is a manufacturer designation, a type designation, an ID, an electrical or geometric nominal parameter, or a material of the ultrasound transmitter, of the ultrasound receiver, or of the channel, or a volume flow, or a type of the fluid, or an operating mode of the detector in a medical treatment or in preparation of a medical treatment.

7. The detector according to claim 1, wherein the ultrasound transmitter and the ultrasound receiver are each formed by a piezoelectric element, wherein an inductance is provided electrically in parallel with the ultrasound transmitter, wherein the pulses of the drive signal comprise a charge pulse having a charging duration for charging the inductance and a number of excitation pulses for exciting the ultrasound transmitter.

8. The detector according to claim 1, wherein the control unit is configured to periodically compare the receive signal with the target value or the target value interval and to adjust the number of and/or the duration of pulses.

9. The detector according to claim 8, wherein the control unit is configured to adjust the number of and/or the duration of pulses when the receive signal leaves the target value interval.

10. The detector according to claim 1, wherein the control unit is configured to increase the number of and/or the duration of pulses when the receive signal lies below the target value or a lower limit of the target value interval, and to reduce the number of and/or the duration of pulses when the receive signal lies above the target value or an upper limit of the target value interval.

11. The detector according to claim 1, wherein the control unit is configured to compare the number of and/or the duration of pulses with a respective predetermined upper limit and to increase the number of and/or the duration of pulses only when the number of and/or the duration of pulses are below their respective predetermined upper limit, such that an upper limit of the transmission energy is not exceeded.

12. The detector according to claim 1, wherein the control unit is configured to compare the number of and/or the duration of pulses with a respective predetermined lower limit and to reduce the number of and/or the duration of pulses only when the number of and/or the duration of pulses exceed their respective predetermined lower limit, such that a lower limit of the transmission energy is not fallen below.

13. A medical treatment device comprising:
a detector according to claim 1; and
a channel arranged between the ultrasound transmitter and the ultrasound receiver,
wherein a fluid is flowable or flows through the channel.

14. A method for detecting inclusions in a fluid flowing extracorporeally using an ultrasound-based detector, the method comprising the steps of:
driving an ultrasound transmitter with a drive signal that comprises pulses in a sequence via a control unit;
transmitting ultrasound with a transmission energy in accordance with the drive signal through a channel conducting the fluid;
providing a receive signal in accordance with a reception energy of the ultrasound via an ultrasound receiver; and
adjusting the transmission energy in order to bring the receive signal to a target value or into a target value interval by means of the control unit,
wherein the step of adjusting the transmission energy is carried out by adjusting a number of and/or a duration of the pulses of the drive signal.

15. The method according to claim 14, further comprising the step of forming a signal derived from the receive signal.

16. The method according to claim 14, further comprising the steps of:
reading at least one parameter of the ultrasound-based detector, of the channel, or of the fluid via the control unit;
accessing a correlation in which an initial number of and/or an initial duration of pulses is or are stored as a function of the at least one parameter via the control unit;
selecting the initial number of and/or the initial duration of pulses; and
driving the ultrasound transmitter with the drive signal having the initial number of and/or the initial duration of pulses, in order to initially bring the receive signal as close as possible to the target value or the target value interval via the control unit.

17. The method according to claim 14, wherein adjusting the number of and/or the duration of pulses is or are carried out until the receive signal has been brought to the target value or into the target value interval via the control unit.

18. The method according to claim 14, further comprising the steps of:
increasing the number of and/or the duration of pulses when the receive signal lies below the target value or below a lower limit of the target value interval; and
reducing the number of and/or the duration of pulses when the receive signal exceeds the target value or exceeds an upper limit of the target value interval via the control unit.

19. The method according to claim 14, further comprising the steps of:
comparing the number of and/or the duration of pulses with a respectively predetermined upper limit;
increasing the number of and/or the duration of pulses only when the number of and/or the duration of pulses are below their respectively predetermined upper limit via the control unit;
comparing the number of and/or the duration of pulses with a respectively predetermined lower limit; and
reducing the number of and/or the duration of pulses only when the number of and/or the duration of pulses are above their respectively predetermined lower limit via the control unit.

20. A non-transitory computer-readable storage medium comprising instructions which, when executed by a computer, cause the computer to carry out the method according to claim 14.
